(19) United States
(12) Patent Application Publication
(10) Pub. No.: US 2022/0395495 A1
(43) Pub. Date: Dec. 15, 2022

(54) SHORTENING TUBERCULOSIS THERAPY AND REDUCING RELAPSE BY CO-ADMINISTERING CHLOROQUINE IN TB AND HIV-TB COINFECTED CONDITIONS

(71) Applicants: FOUNDATION FOR NEGLECTED DISEASE RESEARCH, Bangalore (IN); INDIAN INSTITUTE OF SCIENCE, Bangalore (IN)

(72) Inventors: Amit SINGH, Bangalore (IN); Richa MISHRA, Kolkata (IN); RK SHANDIL, Bangalore (IN); Shridhar NARAYANAN, Bangalore (IN)

(21) Appl. No.: 17/775,285

(22) PCT Filed: Nov. 8, 2020
(86) PCT No.: PCT/IB2020/060500

§ 371 Date: May 7, 2022

(30) Foreign Application Priority Data
Nov. 9, 2019 (IN) 201941045667

(51) Int. Cl. A61K 31/4706; A61K 31/47; A61K 31/454; A61K 31/506; A61K 31/4545; A61K 31/5415; A61K 31/132; A61K 31/498; A61K 31/175; A61K 31/4709; A61K 31/675; A61K 31/5377; A61K 31/541; A61P 31/06

(52) U.S. Cl. CPC A61K 31/4706; A61K 31/47; A61K 31/454; A61K 31/506; A61K 31/4545; A61K 31/5415; A61K 31/132; A61K 31/498; A61K 31/175; A61K 31/4709; A61K 31/675; A61K 31/5377; A61K 31/541; A61P 31/06

(57) ABSTRACT

The present invention provides shortening TB Therapy and reducing relapse by co-administering Chloroquine with anti-TB drugs to drug-sensitive TB patients, multiple drug resistant (MDR) TB patients and TB patients co-infected with HIV-1. The present invention also provides shortening TB Therapy and reducing relapse by co-administering hydroxychloroquine with anti-TB drugs to drug-sensitive TB patients, multiple drug resistant (MDR) TB patients and TB patients co-infected with HIV-1.

### CROSS-REFERENCE TO RELATED APPLICATIONS

This application is a National Stage application of the International Patent Application No. PCT/IB2020/060500, filed 8 Nov. 2020, titled SHORTENING TUBERCULOSIS THERAPY AND REDUCING RELAPSE BY CO-ADMINISTERING CHLOROQUINE IN TB AND HIV-TB COINFECTED CONDITIONS and published as WO 2021/090283, which claims priority to and the benefit of Indian Provisional Patent Application No. 201941045667 filed on 9 Nov. 2019, each of which is incorporated herein by reference in its entirety.

### FIELD OF INVENTION

The present invention is related to shortening TB Therapy and reducing relapse by co-administering Chloroquine with anti-TB drugs to drug-sensitive TB patients, multiple drug resistant (MDR) TB patients and TB patients co-infected with HIV-1.

### BACKGROUND OF THE INVENTION

An unusually long-term therapy (6 months) involving multiple antibiotics is required to cure tuberculosis (TB) in humans. This protracted treatment is necessary to prevent relapses due to genetically drug-sensitive bacteria that become transiently resistant inside host cells and tissues, a phenomenon called phenotypic drug tolerance. Thus, the mechanistic basis of phenotypic drug tolerance needs to be studied to develop new drugs with treatment-shortening properties. Recent studies indicate that heterogeneity in both the host environment and the bacterial population can promote phenotypic drug tolerance. For example, variability in the activation status of macrophages distinctly modulates drug tolerance in Mycobacterium tuberculosis (Mtb) (J Exp Med 2016, 213:809-825). Immune activation of macrophages leads to release of antibacterial effectors such as reactive nitrogen species (RNS) and reactive oxygen species (ROS) (Annual review of immunology 1997, 15:323-350), leading to a quiescent drug-tolerant state of Mtb (Cell Host Microbe 2015, 17:32-46). In support of this theme, drug tolerance is diminished in mice and macrophages deficient in producing nitric oxide (NO). Moreover, extracellular Mtb present in the cavity caseum derived from Mtb-infected rabbits show slow replication and extreme tolerance to several first and second line anti-TB drugs (Antimicrob Agents Chemother 2018, 62: e02266-17). Single cell measurements have revealed that stress conditions (for example, starvation) in vitro and host immune pressures (Interferon-γ, a cytokine critical for anti-TB host immunity) in vivo create phenotypic heterogeneity within the Mtb population, which allows for the selection of non-growing metabolically active bacteria responsible for post-chemotherapeutic relapse (Cell Host Microbe 2015, 17:32-46).

However, recent studies suggest that adoption of a non-growing state is not a prerequisite for drug tolerance (Cell 2011, 145:39-53; Nature 2017, 546:153-157). A fraction of both replicating and non-replicating bacteria show regrowth after drug withdrawal (Science 2013, 339: 91-95), emphasizing that growth-arrested bacteria do not solely mediate tolerance. Alternate mechanisms, such as induction of drug-efflux pumps, asymmetric cell division, and increased mistranslation rates, can contribute to substantial drug tolerance in actively multiplying cells (Science 2012, 335:100-104, Proc Natl Acad Sci USA, 2014, 111:1132-1137). Induction of efflux pumps is, so far, the only mechanism known to confer drug tolerance in replicating Mtb inside macrophages (Cell 2011, 145:39-53). Despite their importance, there is a lack in the understanding of macrophage-specific cue(s) and associated changes in the physiology of replicating Mtb that drive drug tolerance. Filling this knowledge gap will help in developing strategies to target both bacterial and host determinants crucial for mobilizing a drug-tolerant phenotype in vivo.

### BRIEF DESCRIPTION OF THE DRAWINGS

FIGS. 1A, 1B, 1C, 1D, 1E, 1F, and 1G: Chloroquine (CQ) counteracts drug tolerance and reduces relapse in vivo. (1A) Strategy to investigate the efficacy of CQ in reducing tolerance against Isoniazid (Inh) and Rifampicin (Rif) and post-therapeutic relapse in vivo. (1B, 1C) Bacterial CFUs were measured in the lungs at the indicated time points. (1D) Gross pathology of lungs of WT Mtb infected mice at 8 weeks post-treatment across experimental groups. (1E) Hematoxylin and eosin-stained mouse lung sections in various drug treated groups. (1F) Bacterial reduction in Guinea pig lungs following treatment with Inh and CQ combinations. (1G) Hematoxylin and eosin-stained Guinea pig lung sections. Dexamethasone (Dex)-induced reactivation of Mtb from the lungs of BALB/c mice (n=5) post-treatment with Inh alone or a combination of Inh and CQ.

FIGS. 2A, 2B, 2C, 2D, 2E, and 2F. Phagosomal pH and redox heterogeneity drive drug tolerance during HIV-TB co-infection (2A) HIV-1 replication upon stimulation of U1 pro-monocytic cell line with 5 ng/mL PMA; Measurement of percent distribution of redox-diverse fractions following Mtb/Mrx1-roGFP2 infection. (2B) U937 (uninfected HIV-1 control) and (2C) U1 macrophages were stimulated with PMA. (2D) Measurement of percent distribution of redox-diverse fractions following Mtb/Mrx1-roGFP2 infection in U1 macrophages untreated or pre-treated with BafA1. Bacillary load in (2E) U937 and (2F) U1 macrophages, untreated or pre-treated with 10 μM CQ or 10 nM BafA1 and infected with wild type (WT) Mtb for 12 h and exposed to Inh (2.18 μM) or left unexposed for an additional 48 hours.

FIGS. 3A and 3B. Chloroquine combination with HREZ shortens duration of TB therapy. (3A) Methodology. (3B) Untreated control mice; Mice treated with HREZ combination; Mice treated with HREZ and Chloroquine combination showing efficacy of chloroquine.

### BRIEF DESCRIPTION OF THE INVENTION

The present invention discloses
a. Shortening tuberculosis (TB) Therapy and reducing relapse of TB by co-administering Chloroquine with anti-TB drugs to drug-sensitive and drug resistant TB TB patients;
b. Shortening TB Therapy and reducing relapse of TB by co-administering Chloroquine with anti-TB drugs to TB patients co-infected with HIV-1.
The anti-TB drugs as mentioned here include but not limited to Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid and Sutezolid.

In one embodiment, it is also envisaged that hydroxychloroquine can replace chloroquine in shortening tuberculosis (TB) Therapy and reducing relapse of TB by co-administering hydroxyhloroquine with anti-TB drugs to drug-sensitive and drug resistant TB patients; and as well as in TB patients co-infected with HIV-1.

Chloroquine Synergizes with First Line TB Drugs to Enhance Efficacy in TB Infection Models:

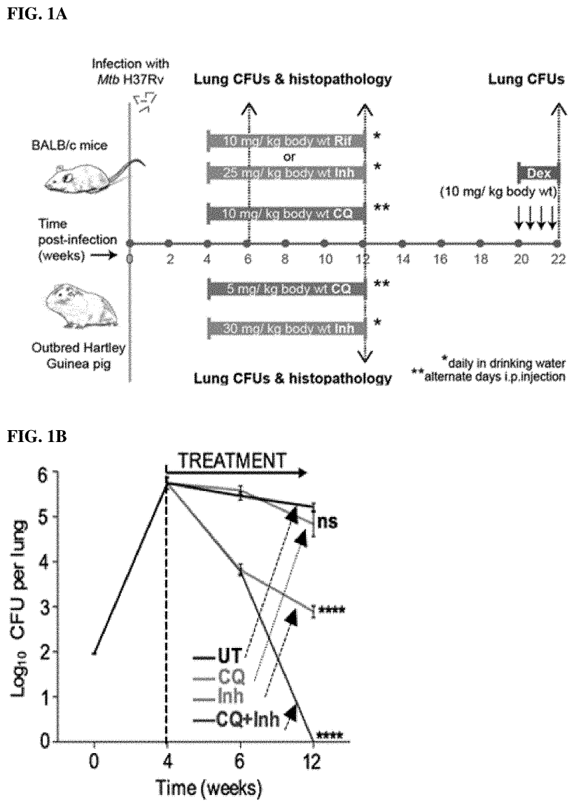

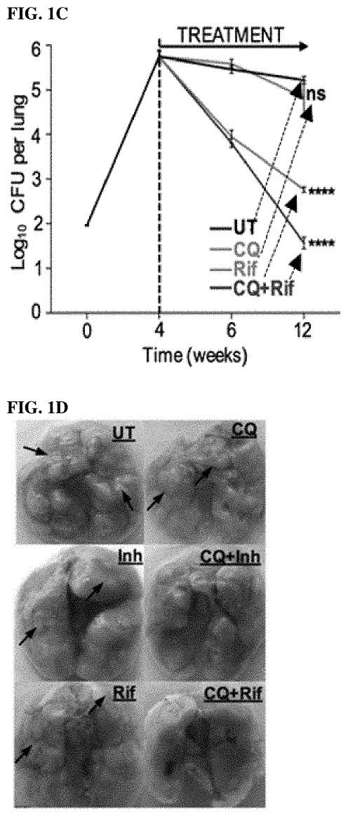

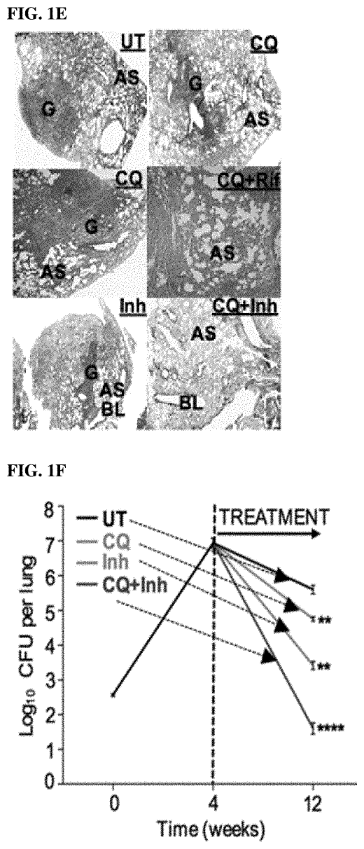

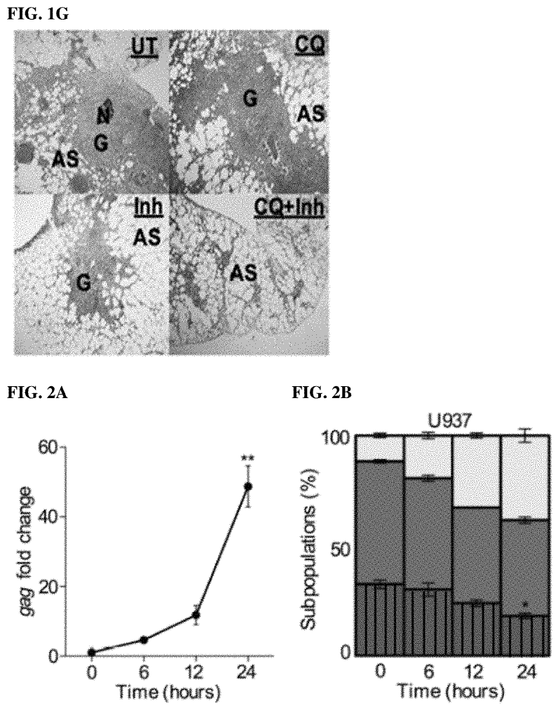

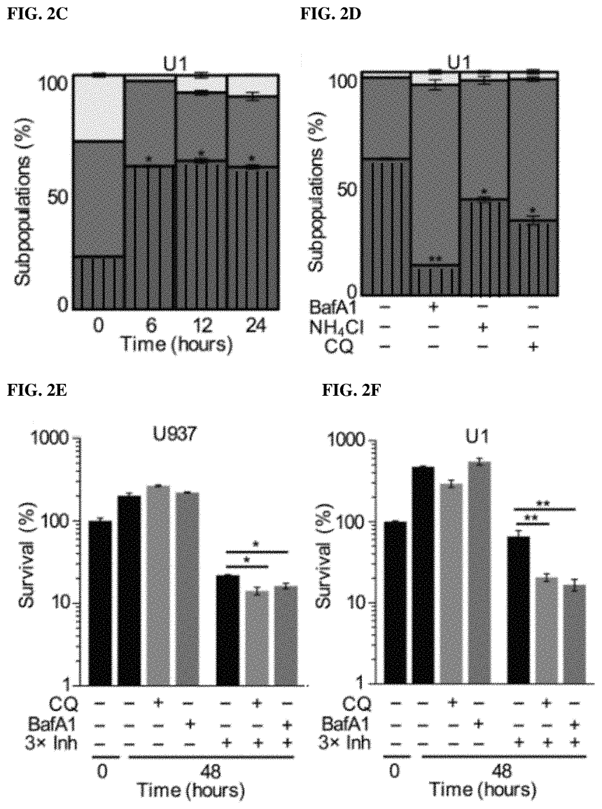

Treatment of TB is exceptionally long and difficult. Treatment of sensitive TB in the clinic takes a minimum of 6 months with a combination of four drugs (isoniazid (H), rifampicin (R), pyrazinamide (Z) and ethambutol (E) commonly termed as HREZ combination) while treatment of multiple drug resistant (MDR) TB takes even 18-24 months with 6-8 second line drugs. The long duration of treatment is toxic and reason for noncompliance and withdrawal in patients that results in therapeutic underexposures and eventually drug resistance. The reason for this longer duration of treatment is the phenotypically drug tolerant population of bacteria that have learned to survive in the acidic macrophage-phagosomal environment. The applicants herein show that antimalarial drug chloroquine (CQ) is a week base and liposomotropic and thus counteracts phenotypic drug tolerance and accelerates killing of Mtb within macrophages by deacidification. In vivo efficacy studies with first line TB drugs-Isoniazid (H) or Rifampicin (R) and Chloroquine combination revealed that addition of 10 mg/kg chloroquine enhanced bacterial killing and efficacy in mice (FIGS. 1B & 1C, respectively) and Guinea pigs (FIG. 1F) TB infection models. Chloroquine though have no or limited direct in vitro activity on TB but it works as an immunomodulator by regulating macrophage phagosomal environment (FIGS. 2A-2F) and synergizes with other TB drugs and results in enhanced bacterial killing in the TB infection models. The present invention thus shows chloroquine synergy/adjuvant activity with TB drugs that can be new repurposed combinations to treat TB.

Sensitive TB is treated with a combination of 4 drugs (HREZ) for 6 months through a Govt. program called directly observed therapy short course (DOTS). Drug resistant TB is much more complex to treat. It usually takes 18-24 months treatment with combination of 6-8 second line drugs. TB drug regimens for humans are largely fixed combinations because they are administered to patients in a DOTS program as catagory1 and 2 combinations depending on susceptibility and resistance patters. All freshly diagnosed patients (unless known case of drug resistance) are given a default category 1 treatment meant for sensitive TB. Catagory1 comprises of Isoniazid, Rifampicin, Ethambutol and Pyrazinamide. (HREZ given for 2 months followed by only HR for next 4 months). All drugs are given either as daily or thrice a week regimen depending on the field/DOTS center resources. TB drugs are generally formulated as DFC's as HREZ or HRE or HRZ or HR only (Daily doses: H75 mg/kg, R150 mg/kg, E275 mg/kg & Z 400 mg/kg). Drugs are adjusted with weight bands covering 30-70 kg. Similarly, combinations with second line drugs or the newer compounds in clinical trials e.g. Bedaquiline, Pretomanid, fluoroquinolones and Linezolid will be appropriately formulated as FDC's or where not possible due to increase in FDC tablet size or physical in compatibility etc will be given as a add on tablet of Chloroquine.

Chloroquine may be formulated as FDC or can be given as a separate tablet with TB drug regimen. Our studies in preclinical models have shown no major drug-drug interaction with HREZ hence CQ can be given simultaneously. TB is a long treatment and Chloroquine may cause retinopathy in few patients by binding to melanin especially when given for months and years long treatment. Hence, chloroquine can be replaced with Hydroxy chloroquine a less toxic metabolite that is widely used in the treatment of many diseases like arthritis, Systemic sclerosis/SLE and Q fever etc.

In one aspect, the present invention provides evidence for shortening duration of TB therapy with chloroquine. Shortening duration of TB treatment is key to its eradication and is the major goal of WHO's global “End TB” strategic targets by 2030. Applicants show that, in mouse TB infection models with SOC TB regimens at human equivalent doses given either alone (H₁₀R₁₀E₁₀₀Z₁₅₀) or in combination with CQ (H₁₀R₁₀E₁₀₀Z₁₅₀+CQ₁₀) for three months to mimic human like TB treatment showed that CQ based combinations are more efficacious than the HREZ alone and completely cleared lung bacterial loads in 3 months versus HREZ treatment that took four months. Thus CQ adjunct therapy facilitates faster killing of TB in the lungs with no detectable bacilli.

Chloroquine doses and route of administration for treatment of TB: In one aspect, Chloroquine is administered intraperitoneally at 10 mg/kg. The route of administration of CQ is by intraperitoneal or oral routes which results in nearly equivalent plasma concentrations and exposures. Standard human doses of CQ in humans vary between 100-300 mg/day depending on clinical indication and duration of therapeutic regimen. Most malaria patients are treated with a loading dose of 600 mg/day and then 300 mg/day on day 2, 3 & 4, that is, total dose of 1500 mg in adults. Malaria prophylactic treatment is 300 mg dose given weekly. Chloroquine is used to treat many other indications for longer durations of months to even years like treatment of Q-fever, arthritis, and endocarditis etc. This also means that CQ long term treatment is safe and clinically tested thus allowing for long term usage for TB treatment as well. These longer chloroquine prescriptions use 300 mg/day for 4 weeks followed by 100 mg/day for months and years.

Chloroquine with TB regimens as FDC or individual tablet/injectable may be given by any route ranging from oral, parenteral, or intravenous injections. However the dose of CQ may vary from 5 mg/day to 300 mg/day depending on its addition to the sensitive or drug resistant TB regimens and duration of treatment. (Since CQ is being repurposed for TB the dose of CQ should be lower than the known human doses (max 300 mg/day for weeks and months) because safety is established over years of clinical practice.

In another aspect of the invention it is envisaged that:

Shortening tuberculosis (TB) Therapy and reducing relapse of TB by co-administering hydroxychloroquine with anti-TB drugs to drug-sensitive TB patients;

Shortening TB Therapy and reducing relapse of TB by co-administering hydroxychloroquine with anti-TB drugs to TB patients co-infected with HIV-1.

The anti-TB drugs as mentioned here include but not limited to Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid and Sutezolid.

Hydroxychloroquine (HCQ), is a soluble beta-hydroxylated derivative of CQ, is relatively less toxic and has been extensively used in the treatment of malaria, connective tissue disorders, and autoimmune and inflammatory conditions such as Rheumatoid arthritis, systemic lupus erythematosus (SLE) (1,2,3,4,5). HCQ is mechanistically similar to CQ with its mode of action involving lysosomotropism, to neutralize acidic endosomes and lysosomes (J Infect Dis 1992, 166(5):1097-1102). Additionally, HCQ administration suppresses pro-inflammatory cytokine signaling (TNFα, IL-6) and its downstream induction of Ca2+-dependent ion channels as well as NLRP3-dependent inflammasome activation (Scientific Reports 2017 volume 7, Article number: 1892). Thus, blocking acidification, suppressing inflammation related signaling and modulating efflux channels. Thus the Applicants propose that HCQ has a role to play in subverting redox heterogeneity in intra-macrophage Mtb, similar to CQ. While the pharmacokinetic parameters for both CQ and HCQ are comparable, HCQ is the clinically preferred therapeutic molecule as it lacks the adverse side-effects observed in case of prolonged CQ administration (Nat Rev Rheumatol. 2018; 14:693-703). HCQ is relatively less retinopathy than CQ for long term use in inflammatory conditions like arthritis and endocarditis (Clin Rev Allergy Immunol, 2012, 42:145-153). Recently, extensive studies have been undertaken to analyze the PK-PD and toxicology parameters associated with the human usage of HCQ even for COVID 19 disease (Infectious Diseases and Therapy, 2020 9:561-572).

Taken together, these features make HCQ a more suitable candidate to repurpose and combine with 1st line ATT, for tackling drug tolerance during Mtb infection. The foregoing examples show the effect of Chloroquine in shortening tuberculosis (TB) Therapy and reducing relapse of TB by co-administering Chloroquine with anti-TB drugs to drug-sensitive TB patients as well as in TB patients co-infected with HIV-1. Due to similar structural and functional attributes Applicants believe hydroxychloroquine will be equally effective under similar situations and conditions with lesser toxicity.

The Examples presented below illustrates and enables the invention. These Examples are for illustrative purpose and should not be construed to limit the scope of the invention.

### EXAMPLES

### Example 1: Chloroquine (CQ) Counteracts Drug Tolerance and Reduces Relapse in Vivo

Mouse infection model of tuberculosis: For the chronic model of infection, 4- to 6-week-old female BALB/c mice (n=6 per group) were infected by the aerosol route with 100 Mtb H37Rv bacilli using a Madison chamber aerosol generation instrument, housed for 4 weeks for progression of infection, and then left untreated or started under various treatment conditions: (i) 10 mg/kg body weight intraperitoneal doses of CQ on alternate days, (ii) 25 mg/kg body weight of Inh in drinking water daily, (iii) 10 mg/kg body weight of Rif in drinking water daily, (iv) a combination of CQ and Inh (CQ plus Inh) at earlier mentioned doses, and (v) a combination of CQ and Rif (CQ plus Rif) at the mentioned doses. At indicated time points of treatment, mice were euthanized, and the lungs were harvested for bacterial burden, gross pathology, and tissue histopathology analysis.

Mice receiving treatment with Inh alone or a combination of CQ and Inh, all treatments were stopped at 12 weeks p.i. (when animals were found to be culture negative for Mtb) for remaining animals (n=5 per group). Animals were further housed for 8 weeks without treatment, after which four intraperitoneal doses of dexamethasone at 10 mg/kg body weight were administered over 2 weeks for pan-immunosuppression. In the 22nd week p.i., animals in both groups were euthanized, and lung burden of reactivated Mtb was determined by plating lung homogenates for CFUs, as mentioned earlier.

Guinea pig infection model of tuberculosis: Outbred Hartley guinea pigs (n=5 per group) were given an aerosol challenge of 100 Mtb H37Rv using a Madison chamber aerosol generation instrument, housed for 4 weeks for progression of infection, and then left untreated or started on treatment in one of three groups: (i) 5 mg/kg body weight intraperitoneal doses of CQ on alternate days, (ii) 30 mg/kg body weight of Inh in drinking water daily, and (iii) a combination of CQ and Inh (CQ plus Inh) at earlier mentioned doses. At 8 weeks after commencement of treatment, guinea pigs were euthanized, and lung burden of Mtb was determined by homogenizing organs in 5 ml of sterile 1 Å˜PBS, serial dilution, and plating on 7H11-OADC agar plates supplemented with PANTA. Upper right lobes of the lungs from different treatment groups were fixed in neutral-buffered formalin and prepared, as mentioned earlier, for histopathological analysis

Results and provided in FIG. 1 and show the following:

FIG. 1: Chloroquine (CQ) counteracts drug tolerance and reduces relapse in vivo. (A) Strategy to investigate the efficacy of CQ in reducing tolerance against Isoniazid (Inh) and Rifampicin (Rif) and post-therapeutic relapse in vivo. BALB/c mice (n=6) were given an aerosol challenge with WT Mtb. From 4 weeks p.i. onwards, groups of mice were left untreated or treated with anti-TB drugs (Inh/Rif) alone or in combination with CQ (Inh+CQ/Rif+CQ). (B, C) Bacterial CFUs were measured in the lungs at the indicated time points. ‘****’p<0.0001 by one way ANOVA with Tukey's HSD correction (untreated vs drug-treated groups). (D) Gross pathology of lungs of WT Mtb infected mice at 8 weeks post-treatment across experimental groups. (E) Hematoxylin and eosin-stained mouse lung sections (8 weeks post-treatment) from mice infected with WT Mtb across experimental groups. The pathology sections show granuloma (G), alveolar space (AS), and bronchiole lumen (BL). All images were taken at ×40 magnification. (F) Bacterial reduction in Guinea pig lungs following treatment with Inh and CQ combinations. Outbred Hartley guinea pigs (n=6) were given aerosol challenge with WT Mtb and efficacy of CQ in reducing Inh tolerance was assessed as described in (B) and (C). ‘**’p<0.01, ‘****’p<0.0001 by one way ANOVA with Tukey's HSD correction (untreated vs drug-treated groups). (G) Hematoxylin and eosin-stained lung sections (8 weeks post-treatment) from guinea pigs infected with WT Mtb across experimental groups. The pathology sections show granuloma (G), alveolar space (AS), and necrotic core (N). Furthermore, Dexamethasone (Dex)-induced reactivation of Mtb from the lungs of BALB/c mice (n=5) post-treatment with Inh alone or a combination of Inh and CQ is shown in Table 1.

TABLE 1
Dexamethasone-based reactivation in treatment groups
of chronically infected BALB/c mice

| Treatment Group | Percentage relapse | Average log₁₀CFU per lung |
|---|---|---|
| Inh alone | 100% (5/5) | 4.2 (n = 5) |
| CQ + Inh | 60% (3/5) | 1.4 (n = 3) |
| | | [p = 0.0069] |

Two-tailed unpaired Student's t-test was used to compare the relapse frequency (Inh alone vs Inh+CQ combination) for effectiveness of CQ therapy. Data shown in each panel are the results of two independent experiments (mean±S.D.). ‘ns’ indicates no significant difference.

### Example 2. Phagosomal pH and Redox Heterogeneity Drive Drug Tolerance During HIV-TB Co-Infection

The pro-monocytic cell lines U937 and U1 were grown similarly, with the exception of 2 mM L-glutamine supplementation for U1 and U937 cells. For all HIV-TB co-infection experiments in these cell lines, U1 cells were differentiated by treatment with 5 ng/mL PMA for 18 h following which immediate infection was carried out, as mentioned earlier. U937 cells were differentiated by PMA treatment at 2.5 ng/mL for 2 days followed by removal of PMA and rest for an additional 24 h for cells to revert to a resting phenotype. PMA-differentiated U1 monocytes were infected at a multiplicity of infection (MOI) of 2 for drug tolerance experiments. After 4 h of incubation with bacteria, infected cells were treated with 0.2 mg/mL amikacin for 2 h, following which infected cells were washed three times with pre-warmed 1×PBS for complete killing and removal of extracellular bacteria. Washed cells were re-incubated in complete RPMI-1640 or DMEM media at 37° C. with 5% CO₂, for indicated time-points of assays. For experiments involving the use of 10 μM chloroquine (CQ), the CQ was added for 1 h prior to infection. Cells were maintained in CQ through the course of the experiments. For colony-forming unit (CFU)-based assays, infected cells were lysed in 0.06% sodium dodecyl sulfate (SDS) in 7H9, diluted as required and plated on 7H11-OADC agar plates. Plates were incubated at 37° C. for 3 weeks before colonies were enumerated. For drug tolerance assays, percent survival at 48 h of treatment with isoniazid was determined by quantifying change in CFUs from 0 h of antibiotic treatment.

Results and provided in FIG. 2 and show the following:

(A) The course of HIV-1 replication upon stimulation of U1 pro-monocytic cell line with 5 ng/mL PMA. Viral load was monitored by gagq RT-PCR. ‘**’p<0.01 by two-tailed unpaired Student's t-test for comparing gag expression with 0 h. (B) U937 (uninfected HIV-1 control) and (C) U1 macrophages were stimulated with PMA, infected with Mtb/Mrx1-roGFP2, and percent distribution of redox-diverse fractions was measured over time. ‘*’p<0.05, ‘**’p<0.01 by two-tailed unpaired Student's t-test. ‘*’ compares E_{MSH}-reduced fraction at various time points with 0 h. (D) U1 macrophages, untreated or pre-treated with 10 nM BafA1, 10 mM NH₄Cl and 10 μM CQ, were infected with Mtb/Mrx1-roGFP2 and percent distribution of redox-diverse fractions was measured at 12 h p.i.‘*’p<0.05, ‘**’p<0.01 by two-tailed unpaired Student's t-test. ‘*’ compares E_{MSH}-reduced fractions between untreated and BafA1/NH₄Cl/CQ treated samples. (E) U937 and (F) U1 macrophages, untreated or pre-treated with 10 μM CQ or 10 nM BafA1, were infected with WT Mtb for 12 h and exposed to Inh (2.18 μM) or left unexposed for an additional 48 h. Bacillary load was determined by CFU enumeration and percent survival was quantified by normalizing the CFU in drug-treated samples at 48 h against untreated samples at 0 h. ‘*’p<0.05, ‘**’p<0.01, ‘***’p<0.001 by two-tailed unpaired Student's t-test. Data shown in each panel are the result of three independent experiments performed in triplicate (mean±S.D.).

### Example 3: Shortening Duration of TB Therapy with Chloroquine

A mouse model of chronic TB infection was used. 4- to 6-week-old female BALB/c mice (n=6 per group) were infected by the aerosol route with 100 Mtb H37Rv bacilli using a Madison chamber aerosol generation instrument, housed for 4 weeks for progression of infection, and then left untreated or treated with either H₁₀R₁₀E₁₀₀Z₁₅₀ or a combination of H₁₀R₁₀E₁₀₀Z₁₅₀+CQ₁₀ orally by gavage. Chloroquine was given intraperitoneally. Treatment started 4 weeks post infection at a chronic TB stage and mice were treated for 3 consecutive months as per design. At indicated time points of treatment, mice were euthanized, and the lungs were harvested for bacterial burden, gross pathology, and tissue histopathology analysis. Data was plotted and the effect of CQ addition to the combination was determined by applying appropriate statistical analysis.

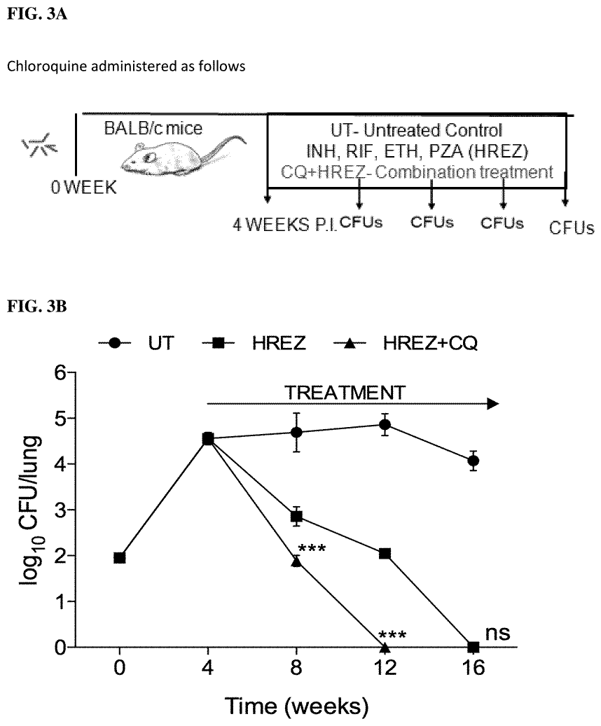

The results show that, in mouse TB infection models with SOC TB regimens at human equivalent doses given either alone (H₁₀R₁₀E₁₀₀Z₁₅₀) or in combination with CQ (H₁₀R₁₀E₁₀₀Z₁₅₀+CQ₁₀) for three months to mimic human like TB treatment showed that CQ based combinations are more efficacious than the HREZ alone and completely cleared lung bacterial loads in 3 months versus HREZ treatment that took four months (FIG. 3). This data thus shows that CQ adjunct therapy facilitates faster killing of TB in the lungs with no detectable bacilli.

1. A method of shortening tuberculosis (TB) therapy in a patient suffering from drug-sensitive TB and drug resistant TB, the method comprising co-administering Chloroquine or hydroxychloroquine with an anti-TB drug.

2. The method of claim 1, wherein the patient is co-administered chloroquine and an anti-TB drug that is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

3. A method of reducing relapse of tuberculosis (TB) in a subject who have recovered from TB, the said-method comprising co-administering Chloroquine with an anti-TB drug.

4. The method of claim 3, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

5. A method of shortening tuberculosis (TB) therapy in a patient co-infected with Human Immunodeficiency Virus-1 (HIV-1) and TB, the method comprising co-administering Chloroquine or hydroxychloroquine with an anti-TB drug.

6. The method of claim 5, wherein the patient is co-administered chloroquine with an anti-TB drug selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

7. The method of claim 3, wherein the patient was co-infected with Human Immunodeficiency Virus-1 (HIV-1) and TB.

8. The method of claim 7, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

9. The method of claim 1, wherein the patient is co-administered hydroxychloroquine with the anti-TB drug.

10. The method of claim 9, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

11. A method of reducing relapse of tuberculosis (TB) in a subject who has recovered from TB, the method comprising co-administering hydroxychloroquine with an anti-TB drug.

12. The method of claim 11, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

13. The method of claim 5, wherein the patient is co-administered hydroxychloroquine with the anti-TB drug.

14. The method of claim 13, wherein the anti-TB drugs is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

15. The method of claim 11, wherein the patient was co-infected with Human Immunodeficiency Virus-1 (HIV-1) and TB.

16. The method of claim 15, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

17. A method of shortening tuberculosis (TB) therapy and reducing relapse of TB in a patients, the method comprises co-administering 5 mg to 300 mg/day of chloroquine or hydroxychloroquine with an anti-TB drug.

18. The method of claim 17, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.

19. The method of claim 17, wherein the patient is co-infected with Human Immunodeficiency Virus-1 (HIV-1) and TB.

20. The method of claim 19, wherein the anti-TB drug is selected from Bedaquiline, Delamanid, TBA 7371, Q203 (Telacebec), BTZ-043, PBTZ-169 (Macozinone), SQ109, Clofazamine, Thiacetazone, Moxifloxacin, SPR-720, Linezolid, and Sutezolid.
